(19) United States
(12) Patent Application Publication
(10) Pub. No.: US 2021/0353929 A1
(43) Pub. Date: Nov. 18, 2021

(54) METHOD AND APPARATUS FOR CLOSING THE UPPER ESOPHAGEAL SPHINCTER DURING ENDOTRACHEAL INTUBATION

(71) Applicant: THE BOARD OF TRUSTEES OF THE LELAND STANFORD JUNIOR UNIVERSITY, Stanford, CA (US)

(72) Inventors: Harmeet S. BEDI, Campbell, CA (US); Dwayne L. FREE, Santa Cruz, CA (US)

(21) Appl. No.: 17/056,835

(22) PCT Filed: May 30, 2019
(86) PCT No.: PCT/US2019/034696

§ 371 Date: Nov. 19, 2020

Related U.S. Application Data
(60) Provisional application No. 62/678,204, filed on May 30, 2018.

(51) Int. Cl. A61N 1/04; A61N 1/36; A61N 1/20; A61M 16/04

(52) U.S. Cl. CPC A61N 1/0452; A61N 1/36003; A61N 1/36007; A61M 16/0465; A61N 1/0492; A61N 1/20; A61N 1/36014

(57) ABSTRACT

A method of closing an upper esophageal sphincter of a patient includes applying electrodes to a neck region of the patient, applying an electric energy through the electrodes such that the upper esophageal sphincter contracts, and deactivating the electric energy, thereby allowing the upper esophageal sphincter to relax. Systems for closing the upper esophageal sphincter are also disclosed.

### CROSS REFERENCE TO RELATED APPLICATIONS

This application claims the benefit of U.S. Provisional Application No. 62/678,204 filed May 30, 2018, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and devices for using electric energy to temporarily close the upper esophageal sphincter of a patient.

### BACKGROUND

Airway securement is a very important procedure that is routinely performed in numerous medical settings. This procedure is commonly accomplished by tracheal or endotracheal intubation, which entails the insertion of a breathing tube through the mouth and into the trachea. Other methods of airway securement involve the insertion of a supraglottic airway device (i.e. any device that sits above the vocal cord region), tracheostomy tube insertion, and cricothyroidotomy. Airway securement is commonly performed in the following situations: procedures requiring sedation, during general anesthesia for procedures and surgeries, patients with declining respiratory function in need of mechanical ventilation support, for airway protection in patients with neurological injury, and in emergency and life-threatening situations. The procedure can be performed by various trained healthcare practitioners (i.e. physicians, respiratory therapists, ambulance personnel, etc.) who have undergone specified training. While not always necessary, prior to airway securement, the practitioner may administer sedatives/anesthetics with or without a neuromuscular relaxant to the patient to allow for a safe and efficient procedure. While these medications are designed to sedate the patient, provide amnesia, and provide a safe environment for the procedure, this also puts the patient into a vulnerable state as these medications can slow-down or stop the patient's breathing. The healthcare practitioner during this time assumes responsibility for the patient's well-being and provides breath/oxygen support by commonly using an oxygen mask and/or bag-valve-mask ventilation.

The safety of this procedure is highly dependent upon the timing and nature of urgency for the procedure. Generally speaking, when this procedure is done in an elective manner (i.e. sedation/anesthesia with a planned procedure/surgery), the procedure is known to be relatively safe. However, if airway securement is required during an urgent or emergent situation (i.e. trauma, declining respiratory status, etc.), the chances of complications dramatically increases. Some of the most dangerous complications related to the procedural method include difficult intubation or airway securement, loss of airway, regurgitation, vomiting, and aspiration. All of these complications can also lead to subsequent need for prolonged mechanical ventilation, hypoxic/anoxic brain injury, comatose state, and even death. Specifically, regurgitation, vomiting, and aspiration during airway securement are known to be catastrophic complications.

One of the most effective methods to prevent the previously stated complications is by advising and confirming that the patient has been “nothing by mouth” or “nil per os” (NPO) for a desired amount of time prior to a procedure/surgery requiring sedation/anesthesia (i.e. at least 6 hours or after midnight). This allows for a minimal amount of food, liquid, or gastric contents to remain in the upper gastrointestinal (GI) tract prior to the airway securement procedure. Hence, there would be no/minimal gastric contents available to be regurgitated, vomited, or aspirated. However, in urgent or emergent situations, there may not be enough time to allow for this preventive process to occur; therefore, these patients would be at an increased risk for such complications. Other solutions/techniques that are utilized to prevent or minimize regurgitation, vomiting, and aspiration include cricoid pressure maneuver (compression of the upper esophagus by pressing on the cricoid cartilage in the neck region), elevating the head of the bed during procedure, pre-procedure nasogastric tube placement, administration of GI stimulant medications, and/or use of certain supraglottic airway devices that are designed to occlude the upper esophagus region.

While the above solutions/techniques currently exist, questions remain about their efficacy in regards to preventing regurgitation, vomiting, and aspiration during the airway securement procedure. According to some studies, aspiration is the leading cause of death from all anesthesia-related complications, and it has a 50-60% mortality rate. Therefore, what are needed and not provided by the prior art are new methods and devices to reduce the complications associated with airway securement.

### SUMMARY

Described herein are new methods, systems and devices to reduce the complications associated with airway securement by using electric energy to temporarily close the upper esophageal sphincter during any airway securement procedure and/or surgery.

According to aspects of the disclosure, a system for closing an upper esophageal sphincter of a patient may be provided with an electrode mount, a plurality of electrodes, at least one lead and a connector. The electrode mount may be configured to engage a neck region of the patient. The plurality of electrodes may be attached to the mount. The at least one lead may extend from the mount and may have a conductor for each of the plurality of electrodes. The connector may be located on a distal end of the at least one lead opposite the mount. The connector may be configured to be connected to an electrical generator. The connector, the conductors and the electrodes may be sized and configured to carry an amount of electrical energy sufficient to substantially fully close the upper esophageal sphincter after general anesthesia induction.

In some embodiments, the conductors and the electrodes are sized and configured to carry between about 100 and about 500 watts. The conductors and the electrodes may be sized and configured to carry between about 20 milliamps and about 5 amps. In some embodiments, the conductors and the electrodes are sized and configured to carry between about 20 and about 50 volts. The conductors and the electrodes may be sized and configured to carry a direct current having a pulse frequency of between about 50 and about 100 hertz. In some embodiments, the pulses have a width that is variable

In some embodiments, the electrode mount includes at least one adhesive patch configured to place at least one of the plurality of electrodes against the neck region of the patient. The electrode mount may include at least one wand configured to place the plurality of electrodes against the neck region of the patient. In some embodiments, the electrode mount comprises a band configured to place the plurality of electrodes against the neck region of the patient. The band may be configured to encircle the neck region of the patient.

In some embodiments, the electrode mount is configured to place the plurality of electrodes directly adjacent to an inferior pharyngeal constrictor muscle. The electrode mount may be configured to place the plurality of electrodes directly adjacent to a cricopharyngeus muscle. In some embodiments, the electrode mount is configured to place the plurality of electrodes directly adjacent to a circular esophageal muscle.

In some embodiments, the system may further include an electrical generator. The electrical generator may be battery powered and weigh less than 50 pounds such that it is portable. In some embodiments, the electrical generator is configured to provide an energy sufficient to constrict the upper esophageal sphincter of the patient before general anesthesia induction.

According to aspects of the disclosure, a method of closing an upper esophageal sphincter of a patient may include the steps of applying a plurality of electrodes to a neck region of the patient, applying an electric energy through the plurality of electrodes such that the upper esophageal sphincter of the patient contracts, and deactivating the electric energy, thereby allowing the upper esophageal sphincter to relax.

In some embodiments, the upper esophageal sphincter contracts into a substantially closed state. The electric energy may be applied for at least 10 seconds. In some embodiments, an endotracheal intubation or supraglottic airway insertion is performed after the esophageal sphincter contracts. The endotracheal intubation or supraglottic airway insertion may be completed before the electric energy is deactivated. In some embodiments, an amperage or voltage of the electric energy is increased during the applying step.

In some embodiments, at least one of the plurality of electrodes is applied directly adjacent to an inferior pharyngeal constrictor muscle. The plurality of electrodes may be applied directly adjacent to a cricopharyngeus muscle.

According to aspects of the disclosure, a system for closing an upper esophageal sphincter of a patient may be provided with an electrode mount, a plurality of electrodes, an electric generator and a plurality of conductors. The electrode mount may be configured to engage a neck region of the patient. The plurality of electrodes may be attached to the mount. The electrical generator may be attached to the mount and configured to provide an energy sufficient to constrict the upper esophageal sphincter of the patient. The plurality of conductors may be electrically interconnecting the plurality of electrodes to the generator. The electrode mount, the plurality of electrodes, the electrical generator and the plurality of conductors together may form a single contiguous unit configured to removably engage the neck region of the patient.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is an anterior view showing typical anatomy of the upper gastrointestinal tract of an adult patient;

FIG. 2A is a lateral view of the neck region of a patient showing the muscles of the pharynx;

FIG. 2B is a partially opened posterior view of the neck region of a patient showing the muscles of the pharynx;

FIG. 3 is a lateral cross-sectional view of a patient's neck showing a prior art method of compressing a portion of the upper esophagus;

FIG. 4A is a photograph showing an upper esophageal sphincter in an open, relaxed state;

FIG. 4B is a photograph depicting the sphincter of FIG. 4A as it would appear after being contracted into a fully closed state through the use of the methods and devices taught by the present disclosure;

FIG. 5A is an anterior view of a patient's neck showing electrode configuration and placement according to one exemplary embodiment of the present disclosure;

FIG. 5B is a lateral view of the patient's neck shown in FIG. 5A;

FIG. 6 is an anterior view of a patient's neck showing electrode configuration and placement according to another exemplary embodiment of the present disclosure;

FIG. 7 is an anterior view of a patient's neck showing electrode configuration and placement according to another exemplary embodiment of the present disclosure;

FIG. 8A is an anterior view of a patient's neck showing electrode configuration, placement, and built-in generator according to another exemplary embodiment of the present disclosure;

FIG. 8B is a lateral view of the patient's neck shown in FIG. 8A;

FIG. 8C is a top view of the combined generator and electrode unit shown in FIGS. 8A and 8B; and

FIG. 9 is a front perspective view showing an electrical generator or power supply unit configured according to an exemplary embodiment of the present disclosure.

### DETAILED DESCRIPTION

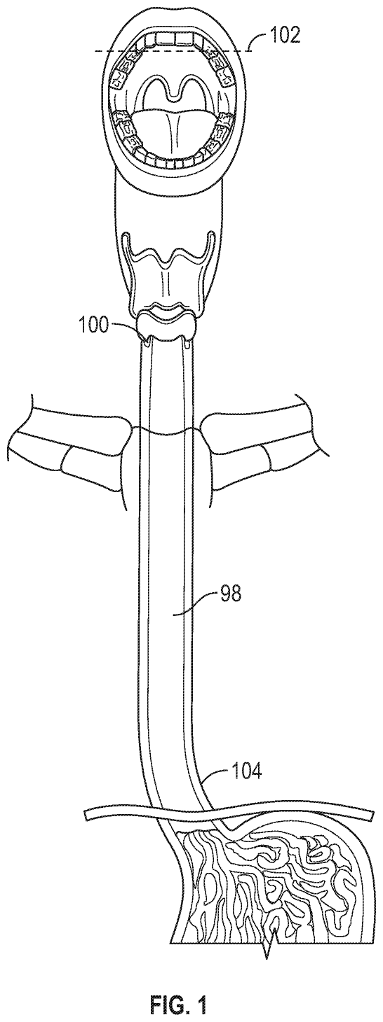

FIG. 1 depicts typical anatomy of the upper gastrointestinal tract of an adult patient, including the esophagus 98. The upper esophageal sphincter 100 is located about 15 cm caudal from the upper palette 102, and about 23 cm cephalad from the lower esophageal sphincter 104.

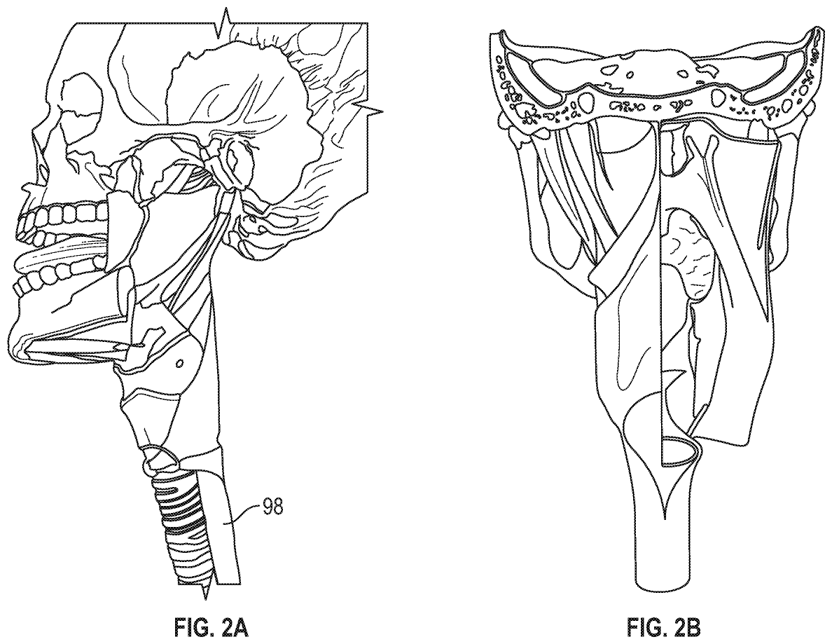

FIGS. 2A and 2B show the muscles of the pharynx, with FIG. 2A providing a lateral view and FIG. 2B providing a partially opened posterior view. As discussed in more detail below, FIGS. 2A and 2B show some of the muscles relevant to electrode placement, according to aspects of the present disclosure.

FIG. 3 depicts a prior art method of compressing a portion of the upper esophagus 98 during airway securement. In this method, a medical practitioner presses on the cricoid cartilage 106 in the neck region 108 of a patient. This cricoid pressure maneuver can help reduce instances of regurgitation, vomiting, and aspiration during an airway securement procedure, but it is not always efficacious, nor without its own side effects and complications.

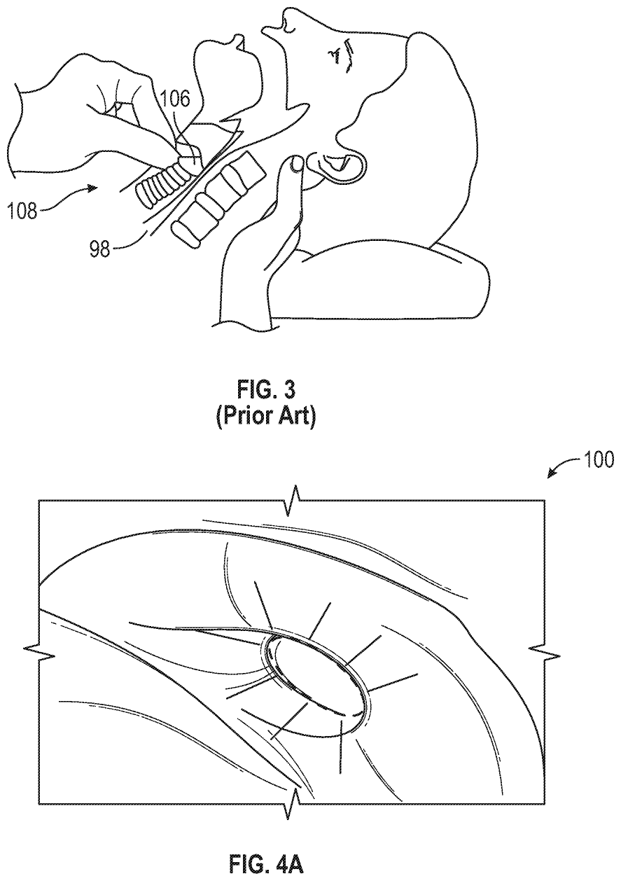

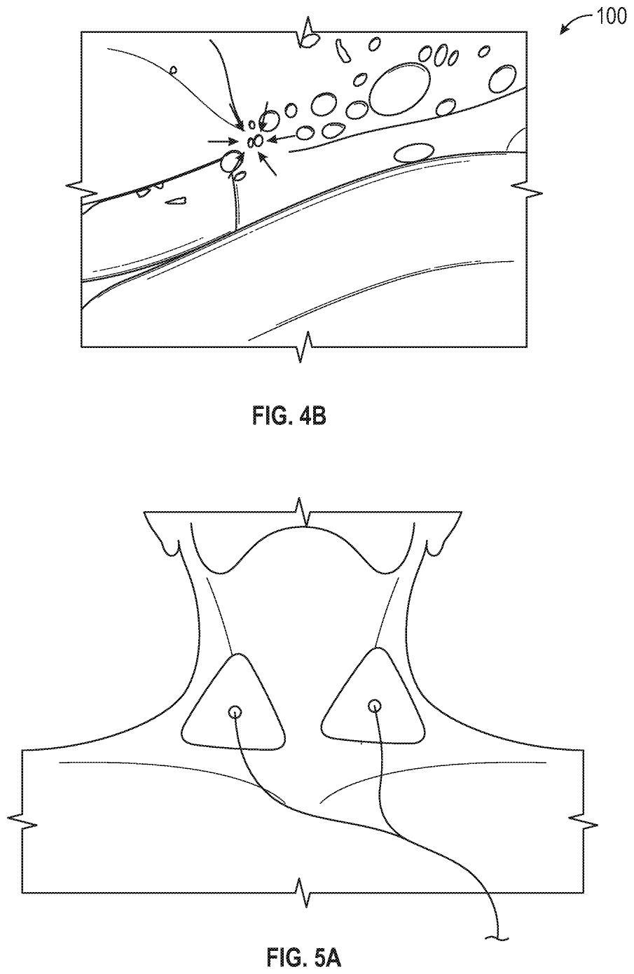

FIG. 4A depicts an upper esophageal sphincter 100 in an open, relaxed state. This is the state in which the upper esophageal sphincter 100 is normally, and would be in prior to application of the electric energy as taught herein. FIG. 4B depicts the sphincter 100 as it would appear after being contracted into a fully closed state through the use of the methods and devices taught by the present disclosure. In some implementations, the sphincter 100 reaches a fully closed state (FIG. 4B) and remains in this state until the electric energy is removed/deactivated. In other implementations, the sphincter 100 reaches a fully closed state (FIG. 4B) upon initial application of electric energy, but starts to relax and move towards a fully open state (FIG. 4A) before the energy is removed/deactivated. In still other implementations, the sphincter 100 moves from an open state (FIG. 4A) to a partially closed state (not shown) when electric energy is applied and never reaches a fully closed state (FIG. 4B).

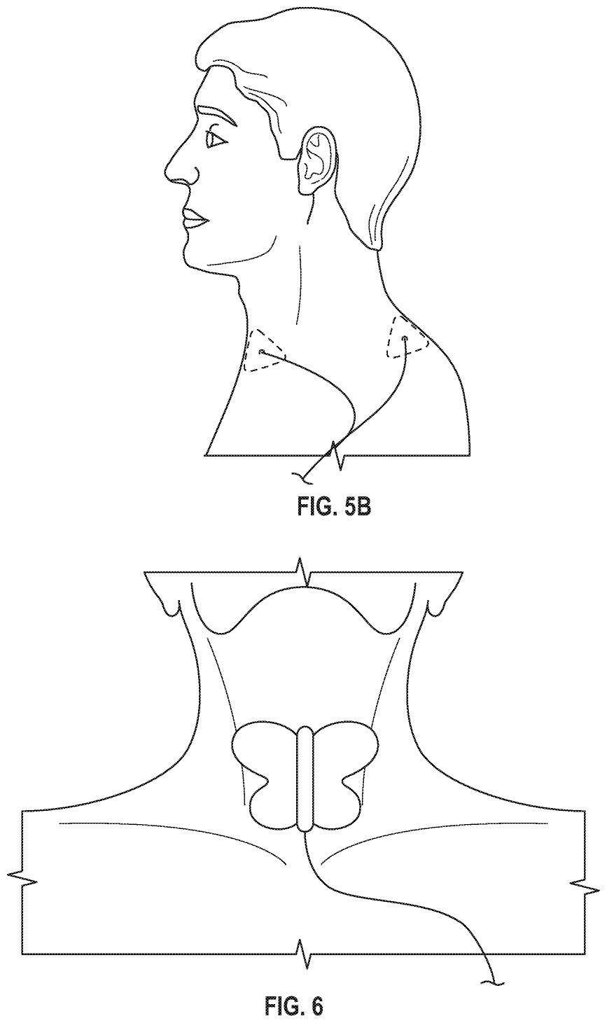

FIGS. 5-8 show various electrode configurations provided according to aspects of the present disclosure. FIGS. 5A and 5B show electrode constructs that may be applied to the neck region via self-adhesive patch(es). In some embodiments (not shown), one or more non-conductive bridges or bands (with or without adhesive) may be provided between the electrode carrying regions of the patch(es) to aid in placement and/or securement of the electrodes. Leads may be provided as shown for connecting the electrode patches to an external electric generator or power supply unit, as will be more fully described below.

FIG. 6 shows a single butterfly-shaped patch for carrying/mounting one or more electrodes.

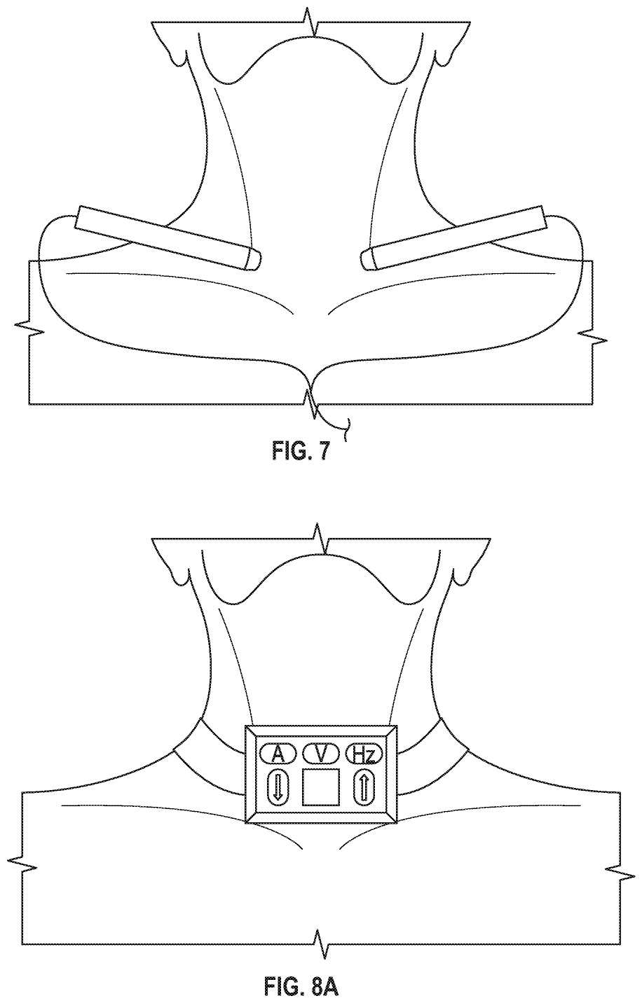

FIG. 7 shows electrodes mounted on a pair of wands that may be manually held against the patient's neck during electric energy application. In other embodiments (not shown), multiple electrodes may be provides on each wand or on a single wand, or a single wand with a single, monopolar electrode may be provided.

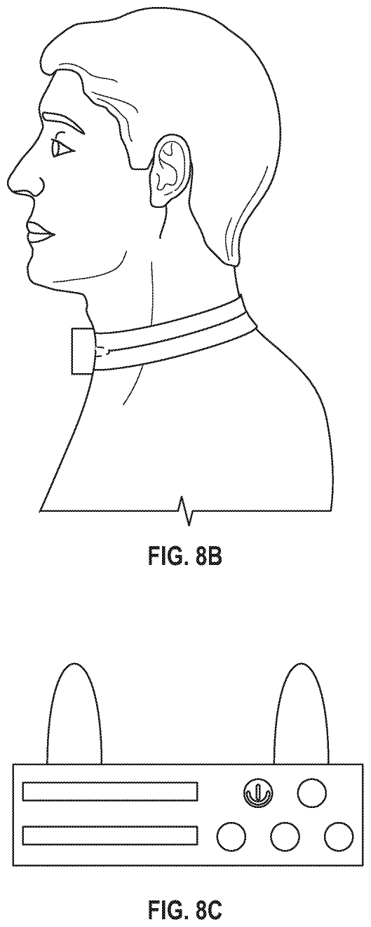

FIGS. 8A-8C show electrodes mounted on a band or collar that is configured to encircle the patient's neck and hold the electrodes firmly against the patient's skin. In some embodiments (not shown), external leads connect the electrodes to a separate energy generator or power supply unit in a similar fashion as the previously described embodiments. Alternatively, as shown in FIGS. 8A-8C, the collar may be a “combined unit” with the electrical generator and the electrode mount combined into a single device. In such combined units, the generator may have built-in electrode(s) on its undersurface. The combined unit may be built in a neck-collar configuration as shown, or into other configurations. The electrodes themselves may be sticky to enhance electrical conduction and securement of the device to the patient's skin and neck surface. The collar adds additional securement. In this exemplary embodiment, the collar has a connector (not shown) to allow both ends to connect securely to each other. The collar material may be fabric and/or synthetic with plastic connectors on the ends.

The electrodes/leads of the combined unit shown in FIGS. 8A-8C may include one or more of the following characteristics:
1. The electrodes/leads may be directly located on the undersurface of the generator and connected by electrical wiring.
2. There may be multiple electrodes/leads located on the undersurface.
3. The electrodes/leads may have prongs or protrusions to allow for indentation of the skin to allow for deeper electrical stimulation and added stability by holding the device in place over the region of the cricoid cartilage.
   Further details of various embodiments of energy generators are provided below.

In other implementations (not shown), other suitable methods (such as a glove or gloves, as subsequently described in more detail) for holding the electrodes against the skin may also be utilized.

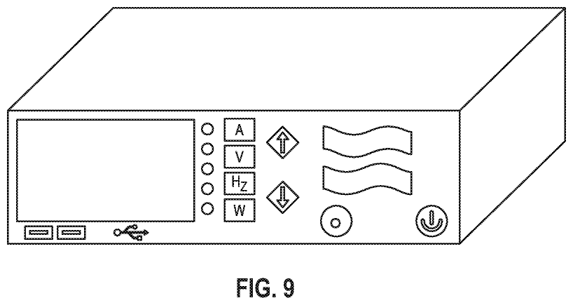

FIG. 9 shows an exemplary standalone energy generator or power supply unit (PSU). Many of the characteristics described in relation to this standalone generator also apply to a generator used in a combined generator and electrode unit, such as previously described in relation to FIGS. 8A-8C. In some implementations, the generator is an adjustable digital switching regulated power supply providing DC power between 0 and 30 volts and 0 and 10 amps. Initial testing has shown that using full power on such a power supply (i.e. 30 volts, 10 amps, 300 watts) is sufficient to close a porcine esophageal sphincter without causing tissue burning or other apparent negative effects. The actual voltage and current reaching the sphincter may be significantly less than those provided by the generator. This is due to resistances encountered by the electric current as it passes through various tissues between the generator electrodes and the sphincter. Once certain tissues have been exposed to anesthesia medicine, electrical resistance can increase significantly. In some embodiments, a potential of between about 20 and about 50 volts may be applied. In some embodiments, a current of between about 20 milliamps and about 5 amps may be applied. In some embodiments, a current of between about 500 milliamps and 3 amps may be applied. In some embodiments, a power of between about 100 and about 500 watts may be applied. In some embodiments, the electric energy applied may be a direct current having pulses. In some embodiments, the pulses have a frequency between about 50 and 100 hertz. In some embodiments, the pulses may have a variable width.

The generator may be a commercially available power supply or equipment that is custom designed for the applications taught in this disclosure. The generator may be operated from an electric outlet (with AC current) or battery operated. The electrical generator may be able to be re-charged via connection to an electric outlet (AC current). In some implementations, the generator is battery powered and weighs less than 50 pounds, making it fully portable. In some implementations, the generator weighs 32 ounces or less. In some implementations, the generator has maximum dimensions of about 12×12×18 inches.

The generator may be adjustable in its settings and/or have pre-programmed power settings available. In other implementations, optimal settings may be predetermined for some or all of the settings indicated below and may not be changeable by the medical practitioner(s) using the system. The following represents different settings that may be adjusted or may be pre-programmed in some implementations:
a. Voltage (V): 0 to 100 V
b. Amperage (A): 0 to 50 A
c. Hertz: 0-100 Hz
d. Watts: 1-1000 Watts
e. Waveform: includes but not limited to:
   i. Square
   ii. Sine wave
   iii. Ramp
   iv. Triangle
   v. Sawtooth

Anticipated anesthesia and patient-specific variables that may be set on the generator include, but are not limited to:
a. Weight based setting (in kilograms)
b. Neck circumference size (in centimeters)
c. Medications given (i.e. whether neuromuscular relaxants are being administered)
d. Urgency (i.e. “Emergency Mode”)

The generator may be provided with one or more of the following feedback confirmation indicators, controls and connectors:
a. Indicators specific to the particular procedure being used.
b. Light illumination/light source indicators/status indicators may be used to indicate if the device has successfully created the intended action at each stage of a procedure.
c. “Activate” button, to initiate desired intended action (e.g. activate current.)
d. “Off” button, to manually turn off the current.
e. Automatic “turn-off” mechanism, to automatically turn off the current after a predetermined period of time has elapsed, such as 2 minutes.
f. Output connector, to which the electrodes/leads may be connected.
g. Grounding pad connector, to which a dedicated grounding pad wire may be connected.

The electrodes/leads may be provided with one or more of the following features:
a. The electrodes/leads may have a connector that is removably attachable to the generator.
b. The length of the wire between the connector and electrodes/leads may be 6 feet.
c. There may be a second wire/lead that acts as a dedicated grounding pad (e.g. with a self-adhesive patch) that connects from the generator to the patient.
   i. One end may have a connector that attaches to the generator.
   ii. The other end may have a sticky lead or patch that adheres to a portion of the patient's body (which may be other than the anterior neck region.)
d. The electrodes or leads may terminate into the following exemplary interfaces. The possible interfaces may have conduction leads built-in to them, to allow for electrical conduction (from the generator to the patient, with a possible feed-back loop).
   i. Electrode patch(es): Leads/pads that adhere to the anterior neck surface, as previously described.
   ii. Wand probes: This may include one or two probes that may be manually held and applied by an operator to the anterior neck surface.
   iii. Collar: This may include a band having electrodes or prongs on the inner surface. The collar may be adjustable in length and circumferentially wrap around the neck. The collar may include a connector to allow wrapping around the neck (e.g. hook and loop type fastener or plastic connector). Additionally, there may be a sticky undersurface to the collar/electrodes that will allow the collar to also adhere to the patient's skin.
   iv. Glove: A single glove or pair of gloves may be configured with built-in electrodes, such as at two or more-digit sites (i.e. thumb and index finger). The glove(s) may be worn by an operator and then pressed against the anterior neck region.
e. The electrodes/leads may be provided in a unipolar and/or bipolar configuration, as well as in a series and/or parallel connection.
f. The electrodes/leads should be capable of carrying the maximum amount of current supplied by the generator (as stated above).
g. The electrodes/leads may be disposable and designed for one-time use.
h. In some implementations, such as some “wand probes” and “collar” interfaces, the electrodes/leads may be designed to be re-usable.

The following methods, steps, and procedures describe exemplary embodiments for using electric energy to temporarily close the upper esophageal sphincter according to aspects of the present disclosure. The instructions provided below are with the assumption that the healthcare practitioner involved has already made the decision to proceed with airway securement in the relevant patient. The following will not address clinical decision-making issues in regards to which patients require an airway securement procedure. Additionally, the following steps are stated with the assumption that the system (and any associated supplies) are already in the vicinity of the patient and healthcare personnel.

The patient in need of an airway securement procedure (i.e. endotracheal intubation or insertion of a supraglottic device) may be physically located in the following locations:
a. Home, commercial location, or roadside (i.e. 911 activation for an emergency medical condition)
b. Ambulance or other medical transport
c. Medical air transport
d. Ambulatory or outpatient clinic
e. Hospital setting
   i. Emergency department
   ii. Intensive care unit and other critical care units
   iii. Medicine wards or standard medical/surgical bed locations
   iv. Operating rooms
   v. Procedure room locations
   vi. Other hospital locations
f. Other location

The healthcare practitioners that may be expected to be involved in the stated procedure include (but not limited to):
a. Licensed physicians
b. Nurse anesthetists—CRNAs (certified registered nurse anesthetists)
c. Nurse practitioners and physician assistants
d. Respiratory therapists
e. Nurses—RNs (registered nurses)
f. Medical assistants
g. Emergency medical technicians (EMTs) or other ambulance/medical transport personnel
h. Trainees of all of the above stated personnel

The following and/or other pre-procedure actions may or may not occur and depend upon specific patient needs. Additionally, the following pre-procedure steps may occur in sequence or concurrently.
a. Oxygen delivery (i.e. via nasal cannula, mask)
b. Venous catheter insertion (central or peripheral) or intravenous (IV) line insertion
c. Cardiorespiratory monitor attachments (i.e. cardiac lead placement, expired end-tidal carbon dioxide monitor, pulse-oximeter, plethysmography, blood pressure cuff placement)
d. Isotonic fluid administration (i.e. normal saline, Ringer's lactate solution)
e. Blood product transfusion
f. Arterial line placement for invasive blood pressure monitoring
g. Right heart catheterization (i.e Swan-Ganz catheter insertion)

Before, during, or immediately after the above stated pre-procedure actions, electrodes and/or leads such as those previously described may be placed on or into the exposed surface of the neck in accordance with aspects of the present disclosure. The electrodes/leads may then be connected to an electrical generator unit, such as those previously described. Alternatively, the leads can be connected to the generator at the time of use. If the “combined collar unit” is used, then the device may be placed around the patient's neck at this time. The estimated time for this step to be completed could range from 1 second to 5 minutes. The regions of the neck that are relevant to electrode/lead placement include but are not limited to:
a. All cartilaginous structures of the neck
   i. Cricoid cartilage
   ii. Superior tracheal rings
b. Pharyngeal, laryngeal, and upper esophageal tissues and/or muscles
   i. Upper esophagus (cervical esophagus)
   ii. Cricopharyngeus muscle
   iii. Inferior pharyngeal constrictor muscle
c. Muscle surrounding or in the vicinity of the above stated structures
   i. Sternocleidomastoid
   ii. Sternothryoid
   iii. Sternohyoid
d. Suprasternal notch and sternum

Next, the airway securement process begins. Practitioners may or may not use sedative, anesthetic, and/or muscle relaxant medications prior to and/or during the actual airway securement procedure. The use and selection of medications is generally based upon practitioner preference, available mediations in the relevant setting, urgency/emergency of airway securement need, and/or patient specific factors. The following are medications that may or may not be used:
a. Sedatives (i.e. benzodiazepines, etomidate, ketamine, barbiturates, etc.)
b. Inhalational anesthetic gases
c. Neuromuscular blockers/relaxants (i.e. succinylcholine, rocuronium, vecuronium, etc.)
d. Other medications

Just prior to the start of the airway securement procedure, the generator (and connected electrodes/leads) may be turned on (or may be on prior to this step). The appropriate generator settings will be selected (or were selected at an earlier time). Once activated, the generator will fire/generate electrical energy or current through the electrodes/leads and cause the intended action (e.g. contraction of the upper esophageal sphincter. If the “combined collar unit” is used, then the device may be activated at this time. In some implementations, the patient must be unconscious prior to activating the device. In some implementations, the generator provides electric energy to the target tissue(s) for between about 1 second to about 5 minutes.

At this point, the practitioner may begin the actual airway securement procedure by either performing an endotracheal intubation or supraglottic airway insertion. The procedural method technique is at the discretion of the proceduralist. In some implementations, the airway insertion procedure takes about 10 to about 30 seconds. In other implementations, the airway insertion procedure can range from about 1 second to about 60 minutes. If the procedure goes perfectly, it can take as little as 3 to 5 seconds. If minor issues are encountered, it can typically take 1 to 2 minutes.

After completion of the airway securement, the generator may be turned off or deactivated. In some implementations, such as when an airway insertion procedure takes more than 5 minutes, the generator may be turned off or deactivated before the airway insertion procedure has been completed. The electrodes/leads may then be removed from the patient. In some implementations, the electrodes and/or leads are designed for single use and are discarded.

The above steps may be performed in a concurrent manner or even out of order, as there may be multiple practitioners attending the patient in these situations. Additionally, the urgency/emergency may affect the sequence or efficacy of some of the steps. In some implementations, it is important that the electrodes/leads are secured to the patient before the generator is activated, in order to provide a consistent, predictable application of energy to the patient and to avoid inadvertently applying energy to medical practitioners. Additionally, in some implementations, it is important that the generator is activated before the airway insertion procedure is commenced. In some implementations, full closure or at least partial closure of the upper esophageal sphincter is confirmed before the airway insertion procedure is commenced. This may be accomplished through imaging of the neck region, through the use of internal or external sensors, or other suitable feedback methodologies.

In some instances, the upper esophageal sphincter may begin to relax after it has been held in a contracted state through the application of electric energy for a period of time. In these instances, an increase in current, voltages or other parameter may be required to hold the sphincter in the contracted state for a longer period of time. In some implementations, the contracted state of the sphincter may be monitored as described above and the electric energy can be increased if and when the monitoring shows the sphincter beginning to open. In some implementations, with or without monitoring, an increasing energy profile may automatically be applied to compensate for the expected relaxation of the sphincter over time. The energy profile may include one or more linear increases, step functions, parabolic, exponential, logarithmic, or other increases, or combinations thereof.

Higher energy levels may be required after anesthesia has been administered to a patient. Controls may be provided on the generator to allow a practitioner to select whether anesthesia has been administered, and/or the type, timing and dosage. In some implementations, a feedback loop may be provided between the anesthesia administration and/or monitoring equipment and the generator to allow the energy level to be adjusted as the anesthesia level changes. In some implementations, one or more control settings may be provided on the generator that reflect characteristics of the patient undergoing the procedure, such as but not limited to weight, neck size, body mass index, age, etc.

While the above discussion is focused on systems and methods for contracting the upper esophageal sphincter during securement of a patient's airway, they may readily be adapted for other portions of this or other procedures. For example, electric energy may be applied to contract the upper esophageal sphincter when an intubation tube is removed from a patient.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the disclosure. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures. is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” or “disclosure” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for closing an upper esophageal sphincter of a patient, the system comprising:
an electrode mount configured to engage a neck region of the patient;
a plurality of electrodes attached to the mount;
at least one lead extending from the mount and having a conductor for each of the plurality of electrodes;
a connector located on a distal end of the at least one lead opposite the mount, the connector configured to be connected to an electrical generator,
wherein the connector, the conductors and the electrodes are sized and configured to carry an amount of electrical energy sufficient to substantially fully close the upper esophageal sphincter after general anesthesia induction.

2. The system of claim 1, wherein the conductors and the electrodes are sized and configured to carry between about 100 and about 500 watts.

3. The system of claim 1, wherein the conductors and the electrodes are sized and configured to carry between about 20 milliamps and about 5 amps.

4. The system of claim 1, wherein the conductors and the electrodes are sized and configured to carry between about 20 and about 50 volts.

5. The system of claim 1, wherein the conductors and the electrodes are sized and configured to carry a direct current having a pulse frequency of between about 50 and about 100 hertz.

6. The system of claim 5, wherein the pulses have a width that is variable.

7. The system of claim 1, wherein the electrode mount comprises at least one adhesive patch configured to place at least one of the plurality of electrodes against the neck region of the patient.

8. The system of claim 1, wherein the electrode mount comprises at least one wand configured to place the plurality of electrodes against the neck region of the patient.

9. The system of claim 1, wherein the electrode mount comprises a band configured to place the plurality of electrodes against the neck region of the patient.

10. The system of claim 9, wherein the band is configured to encircle the neck region of the patient.

11. The system of claim 1, wherein the electrode mount is configured to place the plurality of electrodes directly adjacent to an inferior pharyngeal constrictor muscle.

12. The system of claim 11, wherein the electrode mount is configured to place the plurality of electrodes directly adjacent to a cricopharyngeus muscle.

13. The system of claim 1, wherein the electrode mount is configured to place the plurality of electrodes directly adjacent to a circular esophageal muscle.

14. The system of claim 1, further comprising an electrical generator.

15. The system of claim 14, wherein the electrical generator is battery powered and weighs less than 50 pounds such that it is portable.

16. The system of claim 14, wherein the electrical generator is configured to provide an energy sufficient to constrict the upper esophageal sphincter of the patient before general anesthesia induction.

17. The system of claim 14, wherein the electrical generator is configured to provide an energy sufficient to constrict the upper esophageal sphincter of the patient after general anesthesia induction.

18. A method of closing an upper esophageal sphincter of a patient, the method comprising:
applying a plurality of electrodes to a neck region of the patient;
applying an electric energy through the plurality of electrodes such that the upper esophageal sphincter of the patient contracts; and
deactivating the electric energy, thereby allowing the upper esophageal sphincter to relax.

19. The method of claim 18, wherein the upper esophageal sphincter contracts into a substantially closed state.

20. The method of claim 18, wherein the electric energy is applied for at least 10 seconds.

21. The method of claim 18, further comprising performing an endotracheal intubation or supraglottic airway insertion after the esophageal sphincter contracts.

22. The method of claim 21, wherein the endotracheal intubation or supraglottic airway insertion is completed before the electric energy is deactivated.

23. The method of claim 18, wherein an amperage or voltage of the electric energy is increased during the applying step.

24. The method of claim 18, wherein at least one of the plurality of electrodes is applied directly adjacent to an inferior pharyngeal constrictor muscle.

25. The method of claim 18, wherein at least one of the plurality of electrodes is applied directly adjacent to a cricopharyngeus muscle.

26. A system for closing an upper esophageal sphincter of a patient, the system comprising:
an electrode mount configured to engage a neck region of the patient;
a plurality of electrodes attached to the mount;
an electrical generator attached to the mount and configured to provide an energy sufficient to constrict the upper esophageal sphincter of the patient; and
a plurality of conductors electrically interconnecting the plurality of electrodes to the generator,
wherein the electrode mount, the plurality of electrodes, the electrical generator and the plurality of conductors together form a single contiguous unit configured to removably engage the neck region of the patient.
